(19) United States
(12) Patent Application Publication
(10) Pub. No.: US 2026/0285901 A1
(43) Pub. Date: Sep. 24, 2026

(54) A METHOD FOR PURIFICATION OF EXTRACELLULAR VESICLES OR ENVELOPED VIRUSES BY MEANS OF SIZE EXCLUSION CHROMATOGRAPHY

(71) Applicant: Cytiva BioProcess R&D AB, Uppsala (SE)

(72) Inventors: Jon Lundqvist, Uppsala (SE); Peter Guterstam, Uppsala (SE); Inger Salomonsson, Uppsala (SE)

(21) Appl. No.: 19/476,070

(22) PCT Filed: Apr. 11, 2024
(86) PCT No.: PCT/EP2024/059842

§ 371 Date: Oct. 16, 2025

(30) Foreign Application Priority Data
Apr. 19, 2023 (SE) 2350471-5

(51) Int. Cl. C07K 1/16; B01D 15/34; B01D 61/14; B01J 20/24

(52) U.S. Cl. CPC C07K 1/16; B01D 15/34; B01D 61/145; B01J 20/24

(57) ABSTRACT

A method of purifying extracellular vesicles or enveloped viruses is provided. The method comprises obtaining a solution volume comprising extracellular vesicles or enveloped viruses and contaminants, adding the solution volume to a size exclusion chromatography column comprising a stationary phase comprising a packed bed of cross-linked polysaccharide beads, and collecting an eluate volume comprising the extracellular vesicles or enveloped viruses exiting the column. Advantageously, the method is scalable and can be used in large scale or industrial scale processes.

### TECHNICAL FIELD

The present invention relates to a method for purification of extracellular vesicles (EVs) or enveloped viruses by means of size exclusion chromatography.

### BACKGROUND ART

Extracellular vesicles (EVs) are lipid bound vesicles secreted by cells into the extracellular space. The three main subtypes of EVs are microvesicles, exosomes, and apoptotic bodies, which are differentiated based upon their biogenesis, release pathways, size, content, and function. Exosomes are present in almost all cells and tissues, and help in intercellular signalling and maintains tissue homeostasis in the disease pathology. Exosomes are composed of cellular membranes with multiple adhesive proteins on their surface and interact with the recipient cells through their surface receptor molecules and ligands and internalize within the recipient cells through micropinocytosis and phagocytosis. Exosomes are gaining increasing interest as cell-free drug-delivery systems, due to their characteristics derived from the parent cell or host cell. The therapeutic potential of exosomes in various diseases, such as cancer, neurodegenerative diseases, cardiovascular diseases and orthopedic diseases is a growing field of research in regenerative medicine.

Exosomes are challenging to purify/isolate as they are vulnerable to shear stress, are large heterogeneous particles (size range about 30-150 nm) and their size overlaps with contaminants from which to be separated. Large scale production of exosomes for use in e.g. therapies is increasing. This also puts a demand on methods for effective and time-efficient purification of exosomes on a large scale.

Enveloped viruses, such as retroviruses, are in some ways similar to EVs as they are in the same size range and are surrounded by a lipid membrane that also contains cells and membrane proteins.

Present known separation techniques for purifying enveloped viruses and extracellular vesicle, such as exosomes, are, however, not very successful for use in large-scale production.

For analytical/diagnostic purposes, commercially available purification kits, ion exchange chromatography and centrifugation techniques may be used for EV or enveloped virus purification. Differential centrifugation separates EVs or enveloped viruses from other components based on size and density differences. Centrifugation may harm vesicles/viruses and is not scalable. Affinity/immuno-capture methods may also be used and are based on interactions with specific EV/virus surface molecules. The above mentioned methods are, however, not scalable or not suitable for large scale purification due to costs. The methods are also time consuming, show poor repeatability, and may harm the EVs/enveloped viruses.

Despite numerous studies, isolating pure preparations of EVs or enveloped viruses has proven challenging. There, hence, remains a demand for large-scale purification techniques, which provide pure isolated EVs or enveloped viruses with a low level of contaminants and which do not harm the EVs/viruses.

### SUMMARY OF THE INVENTION

It is an object of the present disclosure to provide a method for purification of extracellular vesicles (EVs) or enveloped viruses by means of size exclusion chromatography.

According to a first aspect, there is provided a method of purifying extracellular vesicles or enveloped viruses, comprising: obtaining a solution volume comprising extracellular vesicles or enveloped viruses and contaminants, adding the solution volume to a size exclusion chromatography column comprising a stationary phase comprising a packed bed of cross-linked polysaccharide beads, and collecting an eluate volume comprising the extracellular vesicles or enveloped viruses exiting the column.

The size exclusion chromatography column may include formats tailored for robotic high-throughput applications.

The polysaccharide beads may be agarose beads. Agarose beads of the packed bed may comprise 0.5-20 wt. % agarose in water, ethanol or a mixture thereof. The agarose content may be 0.5-20 wt. %, 1.5-20 wt. %, 2-20 wt. %, >2-20 wt. %, 2.5-20 wt. %, 3-20 wt. %, 3.5-20 wt. %, 4-20 wt. %, 4.5-20 wt. %, 5-20 wt. %, 5.5-20 wt. %, 6-20 wt. %, 6.5-20 wt. %, 7-20 wt. %, 7.5-20 wt. %, 8-20 wt. %, 8.5-20 wt. %, 9-20 wt. %, 9.5-20 wt. %, 10-20 wt. %, 11-20 wt. %, 12-20 wt. %, 13-20 wt. %, 14-20 wt. %, 15-20 wt. %, 16-20 wt. %, 17-20 wt. %, 18-20 wt. %, 19-20 wt. %, 3-10 wt. %, 5-15 wt. %, or 10-20 wt. %.

The polysaccharide beads are cross-linked. Cross-linking may allow for use of higher flows through the column. In the case of agarose beads, the higher the initial agarose concentration, the smaller the pore sizes that form after cross-linking. The beads are porous beads that contain pores of a defined size range. Polysaccharide beads, such as agarose beads, useful in the present invention can be produced e.g. as described in U.S. Pat. Nos. 6,602,990 B1, 7,396,467B2 or 8,309,709 B2, which are incorporated herein.

The bead size D50v may be 5-200 μm, wherein D50v is the median particle size of the cumulative volume distribution. Smaller particles than 5 μm may result in high pack-pressure and column-nets, i.e. leakage of small beads through the column filter. The bead size D50v may be 5-200 μm, 10-200 μm, 20-200 μm, 40-200 μm, 60-200 μm, 80-200 μm, 100-200 μm, 120-200 μm, 140-200 μm, 160-200 μm, 180-200 μm, 5-180 μm, 5-160 μm, 5-140 μm, 5-120 μm, 5-100 μm, 5-80 μm, 5-60 μm, 5-40 μm, 5-20 μm, or 10-50 μm.

The present beads can be packed in the column at a significantly higher flow than what is used in conventional packing, i.e. gravitational settlement (>30 cm/hour). The cross-linked beads are compatible with packing in columns at >120 cm/h at bed heights >5 cm. The rigidity of such formed size exclusion stationary phase allows chromatography to be performed at flow velocities up to 700 cm/hour. Chromatography performed at a high flow improves the productivity in the manufacturing of EVs or other biological particles. Column packing at a flow higher than gravitational settlement is required, at large-scale, to efficiently achieve a well-packed stationary phase, meeting potential criteria for plate height and asymmetry factor.

The present beads may have an exclusion limit of at least 30 nm, or at least 40 nm, which means that biological particles having a size above this size limit, such as exosomes, will not diffuse into the pores of the beads and be retained in the column. Biological particles with sizes larger than the exclusion limit do not enter the pores and pass through the packed column relatively quickly by making their way between the beads. Smaller biological particles, which can enter the pores of the beads, do so, and thus, have a longer path that they take in passing through the packed bed in the column. Thereby, biological particles larger than the exclusion limit, i.e. the extracellular vesicles such as exosomes (size range about 30-150 nm) or enveloped viruses (size range about 30-300 nm) will leave the column earlier, while smaller biological particles will pass through the beads and will elute from the column later. All analytes in the sample that are equal to, or larger, than this critical size will behave identically: they will all be eluted in the excluded volume of the column.

In size exclusion chromatography (SEC) one or more compounds in a mixture are concentrated or isolated. The compounds in the mixture traverse the chromatographic column at different rates, leading to their separation. The migration occurs by convection of a fluid phase, the mobile phase, in relationship to the packed bed of beads, the stationary phase. In SEC the analytes in a mixture are separated or isolated on the basis of hydrodynamic radius. As EVs/enveloped viruses are spherical the hydrodynamic radius can be related to the biological particle size in nm. In SEC separation occurs because of the differences in the ability of analytes to probe the volume of the porous stationary phase media. SEC is typically used for the separation of large biological particles or complexes of biological particles, such as exosomes, deoxyribonucleic acid (DNA), ribonucleic acid (RNAs), proteins, polysaccharides and fragments and complexes thereof for analytical purposes. The sample mixture is loaded onto the SEC column under pressure and the mixture and mobile phase are pushed through the column. The compounds in the mixture leave or elute from the column with the largest compounds exiting first and the smallest biological particles leaving last.

The column may be placed in fluid communication with a detector, which can detect the change in the nature of the solution as the solution exits the column. The detector will register and record these changes as a plot, referred to as a chromatogram, which is used to determine the presence or absence of the analyte. The time at which the analyte leaves the column is an indication of the size of the biological particle. Molecular weight of the biological particles can be estimated using standard calibration curves, Nano tracking analysis (NTA) or e.g. by using light-scattering (MALS) on-line, mass spectrometry etc. Size exclusion does not rely on any chemical interaction between analyte and the stationary phase, rather it is based on a physical property of the analyte, here the extracellular vesicles (EVs) or enveloped virus, to be purified, i.e. the effective particular radius (which relates to mass for most molecules). The chromatography column may be used for purifying EVs/enveloped viruses in a solution also comprising contaminants such as host cell DNA, proteins and endotoxins.

Enveloped viruses and EVs, such as exosomes, are challenging to purify/isolate as they are vulnerable to shear stress, are large heterogeneous particles (size range about 30-300 nm) and their size overlaps with contaminants from which to be separated. Large-scale production of exosomes for use in e.g. therapies is increasing. This also puts a demand on methods for effective and time-efficient purification of EVs/enveloped viruses on a large scale.

Techniques that can be used mainly for analytical purification of EVs or enveloped viruses are ion exchange chromatography and centrifugation techniques, which separates EVs/viruses from other components based on size and density differences. Centrifugation, however, may harm the vesicles/viruses and is not easily scalable. Affinity/immuno-capture methods can also be used on an analytical scale, but are not suitable for large-scale purification due to high costs. The above-described size exclusion chromatography column can be used to purify extracellular vesicles (such as exosomes)/enveloped viruses from smaller sized impurities. The described size exclusion chromatography stationary phase has such flow properties that it is suitable for packing in large-scale columns. Mild buffer conditions may be used, and the vesicles/viruses are subject to low shear stress, keeping the isolated/separated vesicles/viruses intact. Any buffer that keeps the integrity of a large bio-entity like extracellular vesicle/enveloped virus may be used.

The method of purifying extracellular vesicles or enveloped viruses described above is scalable, i.e. the same process parameters can be employed using equipment and consumables of various sizes or volumes, including large scale or industrial size equipment. For the method described above, with a size-exclusion chromatography column comprising a packed bed of cross-linked polysaccharide beads, it means that the same flow and other process parameters can be employed in a chromatography column with 0.5 cm diameter and a large column with e.g. 60 cm diameter. The described cross-linked beads are compatible with packing in such large-scale columns.

Before adding the solution volume comprising extracellular vesicles or enveloped viruses to the size exclusion chromatography column, there may be a step of filtering the solution volume comprising the extracellular vesicles/enveloped viruses with a filter or membrane to remove contaminants being of a size smaller than the extracellular vesicles from the sample volume.

In the filtration step, most of the contaminants being of a smaller size than the enveloped viruses/EVs, such as smaller proteins, are removed from the solution volume.

The filter/membrane used may for example have a cut-off of 300-750 kDa, preferably 750 kDa. A cut-off of 750 kDa is suitable when purifying extracellular vesicles such as exosomes. The filtering step may comprise tangential flow filtration (TFF), in which the solution volume comprising the EVs or enveloped viruses and any contaminant is passed across a filter membrane at a positive pressure relative to permeate side to remove contaminants. Before such filtering step there may be a clarification step in which the solution volume is passed through a filter having a pore size of for example 0.22 μm or 0.4 μm to remove larger particles or microorganisms from the solution volume.

The method may further comprise concentration of the filtered solution volume.

The solution volume comprising the extracellular vesicles or enveloped viruses may be concentrated to a smaller volume before being added to the size exclusion chromatography column. The solution volume may be concentrated for example 10 times, or 20 times.

Due to the concentration step, a chromatography column with less packed bed volume may be used than if non-concentrated solution volumes are added to the chromatography column. Also, the concentration step results in a more concentrated eluate solution comprising the extracellular vesicles or enveloped viruses exiting the chromatography column. The concentration step may be performed by TFF directly after or during the filtering step above.

Before or after filtering the solution volume, a nuclease, in particular a deoxyribonuclease (DNase), may be added to the solution volume.

In a subsequent filtering, digested DNA is removed from the solution volume. The DNase may be kryptonase, benzonase, etc. The nuclease digests large DNA impurities to smaller pieces (smaller than the size of the EVs) that can be removed by filtering, such as by TFF. Thereby, less contaminants remain in the solution volume when the solution volume is added to the chromatography column.

The solution volume may be added to the size exclusion chromatography column at a flow velocity of at least 30 cm/hour.

The flow velocity may be at least 30 cm/hour, or 30-700 cm/hour, or 50-700 cm/hour, or 100-700 cm/hour, or 120-700 cm/hour, or 150-700 cm/hour, 175-700 cm/hour, 200-700 cm/hour, 250-700 cm/hour, 300-700 cm/hour, 350-700 cm/hour, 400-700 cm/hour, 450-700 cm/hour, 500-700 cm/hour, 550-700 cm/hour, 600-700 cm/hour, 650-700 cm/hour, 300-650 cm/hour, 30-600 cm/hour, 30-550 cm/hour, 30-500 cm/hour, 30-450 cm/hour, 30-400 cm/hour, 30-350 cm/hour, 30-300 cm/hour, 30-250 cm/hour, 30-200 cm/hour, 30-175 cm/hour, 30-150 cm/hour, 120-500 cm/hour, 200-400 cm/hour, 400-600 cm/hour, or 500-700 cm/hour. Under these flow velocity conditions, a short residence time is obtained, which is important for a fast purification process, which is of particular interest for large-scale purification processes. These method conditions also enable the use of chromatography columns having a packed bed with a height of 20 cm or less, the packed bed volume depending on column diameter. Using a flow velocity of up to 700 cm/h, a residence time of as little as 1 min may be obtained. The flow velocity depends on the column length, i.e. back pressure. The gravity flow velocity is approximately 30 cm/hour.

Compared to conventional size exclusion chromatography columns, which have a flow velocity limit around 120 cm/h, the present size exclusion chromatography column packed bed of cross-linked agarose beads can be used with much higher flow velocities and, hence, shorter residence times.

The method provides for a way of separating enveloped viruses/extracellular vesicles, such as exosomes, leaving a low level of contaminants in the solution, wherein the vesicles/viruses are subject to low shear stress, keeping the isolated/separated vesicles/viruses intact.

The solution volume may be added to the size exclusion chromatography column at a pressure of at most 3 bar.

The solution volume may be added to/loaded to the column at a pressure of up to 3 bar. Use of such high pressures provides for extracellular vesicle/enveloped viruses separation, wherein the vesicles/viruses are subject to low shear stress, keeping the isolated/separated vesicles/viruses intact.

The solution volume comprising extracellular vesicles or enveloped viruses may be added to the column in a ratio of solution volume to column/packed bed volume of up to 1:3.

The larger the volume of the solution added, the more overlap between separated molecules coming out from the column. The sample volume added may be up to 1:3, or 1:4 or 1:5 of the column volume. Larger sample volumes imply risk of limited separation of small-sized impurities from large bio-entity pool. Large sample volumes means that fewer chromatography cycles are needed to purify the sample/feed volume.

The packed bed of polysaccharide beads may have a height of 2.5-60 cm.

The chromatography column may have a width of 0.5 cm to 70 cm in diameter.

Columns with a diameter larger than 5 cm can be considered large, such as 5-70 cm, or 5-60 cm, or 10-60 cm, or 20-60 cm. The bed height of such large-scale columns may be in the range of 2.5-60 cm, or more commonly 10-30 cm. The packed bed of cross-linked agarose beads may have a height of 10-60 cm, or 15-60 cm or 20-60 cm.

In size exclusion chromatography, the resolution is a function of column length/packed bed height (the longer the better) and flow rate (the lower flow rate, the better resolution).

The purification method may be used for purification of exosomes.

### BRIEF DESCRIPTION OF THE DRAWINGS

FIG. 1 shows residence times obtained for size exclusion chromatography (SEC) columns run at different flow velocities (100-700 cm/h). Three different bed heights (10 cm, 15 cm and 20 cm) of columns having a stationary phase comprising a packed bed of highly cross-linked agarose beads were compared. A reference column comprising a packed bed of Sepharose® CL-2B with a bed-height of 10 cm was used. The figure shows that the resolution when using the highly cross-linked agarose packed bed is in line with or superior to Sepharose® CL-2B.

FIG. 2 is a chromatogram showing a comparison of the eluate from a (SEC) column comprising a highly cross-linked agarose stationary phase (curve 1, A214 nm, curve 4, A490 nm) with the eluate from a SEC column comprising a Sepharose® CL2-B stationary phase (curve 2, A214 nm, curve 3, A490 nm) (bed height 10 cm), the columns being loaded with a solution volume comprising a mixture of latex particles (60 nm) and proteins (GFP-His, 28 kDa) at a flow velocity of 129 cm/h and evaluated with absorbance spectroscopy A214 nm and A490 nm.

FIG. 3 shows an overlay of two chromatograms obtained from a SEC column comprising a highly cross-linked agarose stationary phase (bed height 10 cm) and being loaded with solution volumes comprising latex particles (60 nm, curve 1) and albumin (69 kDa, 3.8 nm, curve 2), respectively, at a flow velocity of 129 cm/h. The eluate was evaluated with absorbance spectroscopy A280 nm (curve 1 and 2) and conductivity (curve 3, peak shows the total column volume of the column where salt is eluted).

FIG. 4 is a chromatogram obtained from a SEC column comprising a highly cross-linked agarose stationary phase (bed-height 10 cm) being loaded with a solution volume comprising a mixture of GFP-exosomes sample spiked with thyroglobulin (669 kDa) at a flow velocity of 129 cm/h. The eluate was evaluated with absorbance spectroscopy A280 nm (curve 1) and 490 nm (curve 2). Conductivity (curve 3, peak shows the total column volume of the column where salt is eluted) is also shown.

FIG. 5a is a chromatogram obtained from a SEC column comprising a highly cross-linked agarose stationary phase (bed-height 10 cm) being loaded with an exosome feed, which had been diafiltered and concentrated (including DNase treatment) with tangential flow filtration (TFF) with a cut-off of 750 KDa before being added to the SEC at a flow velocity of 129 cm/h and evaluated with absorbance spectroscopy at A280 nm. FIG. 5b shows detection of exosomes in different eluate fractions using ELISA CD63 (marker for exosomes) detection (line) and total protein concentration using bicinchoninic acid assay (BCA assay) (bars).

FIG. 6a is a chromatogram obtained from a SEC column comprising a highly cross-linked agarose stationary phase (bed-height 20 cm, bed volume 4.7 ml) being loaded with an exosome feed, which had been diafiltered and concentrated (including DNase treatment) with tangential flow filtration (TFF) with a cut-off of 750 KDa before being added to the SEC at a flow velocity of 129 cm/h evaluated with absorbance spectroscopy at A280 nm. FIG. 6b shows detection of exosomes in different eluate fractions using ELISA CD63 detection (line) and BCA assay (bars).

FIGS. 7a and 7b show the particle concentration in different fractions eluted from the columns discussed for FIGS. 5a-5b and FIGS. 6a-6b, respectively. The concentration was measured using nanoparticle tracking analysis (NTA) (ZETAVIEW® from Particle Metrix).

FIGS. 8a and 8b show the particle size in different fractions eluted from the columns discussed for FIGS. 5a-5b and FIGS. 6a-6b, respectively. The particle size was measured using nanoparticle tracking analysis (NTA) (ZETAVIEW® from Particle Metrix). X10=particle size where 10% of the particles are smaller than that particle size (nm), X50=particle size where 50% of the particles are smaller than that particle size (nm) and X90=particle size where 90% of the particles are smaller than that particle size (nm).

FIG. 9a is a chromatogram obtained from a SEC column with a highly cross-linked agarose packed bed (bed height 10 cm) and fed at a flow velocity of 129 cm/h with a harvest comprising exosomes produced by a HEK (Human Embryonic Kidney) 293 cell line. FIG. 9b is a chromatogram obtained from a SEC column with a highly cross-linked packed bed of agarose beads (bed height 10 cm) and fed at a flow velocity of 129 cm/h with a harvest comprising exosomes produced by a HEK (Human Embryonic Kidney) 293 cell line, which harvest before being applied to the SEC column had been diafiltered and concentrated (including DNAse treatment) with a TFF with a cut-off of 750 KDa. FIG. 9c shows the total protein concentration in different fractions eluted from the columns discussed for FIGS. 9a (no TFF) and 9b (with TFF) using the BCA assay. FIG. 9d shows the exosome content in different fractions eluted from the columns discussed for FIGS. 9a (no TFF) and 9b (with TFF) using the ELISA CD81 assay.

FIG. 10 shows an overlay of the chromatograms shown in FIG. 9a and FIG. 9b.

FIG. 11a shows the total protein content as measured with the BCA assay, and in FIG. 11b is shown the exosome content as measured with ELISA CD63 in different eluate fractions from SEC columns having a stationary phase of highly cross-linked agarose and Sepharose®-2L-B, respectively (bed height 10 cm). The columns being fed at a flow velocity of 129 cm/h with a harvest comprising exosomes produced by a dental pulp cell line. The graph shows the result with and without a preceding diafiltering step and 9.3× concentration with TFF with a cut-off of 750 kDa. The sample (harvest) was treated with DNase (benzonase) before being filtered and concentrated in the TFF step.

FIG. 12 shows a chromatogram obtained from a highly cross-linked agarose SEC column (10 cm bed height) loaded at a flow velocity of 129 cm/h with a sample containing GFP-exosomes spiked with thyroglobulin (680 KDa) as measured with A490 nm and A280 nm.

FIG. 13a shows a chromatogram obtained from a SEC column with a packed bed of highly cross-linked agarose beads (bed-height 20 cm) and fed at a flow velocity of 38 cm/h with an exosome feed which had first been diafiltered and concentrated (including DNase treatment) with TFF (cut-off 750 kDa). (Bed volume was 17 ml). FIG. 13b shows the total particle content (line) as measured with the BCA assay in different eluate fractions, and the particle concentration (bars) in different eluate fractions measured using nanoparticle tracking analysis (NTA) (ZETAVIEW® from Particle Metrix).

FIG. 14a shows a chromatogram obtained from a SEC column with a packed bed of highly cross-linked agarose beads (bed-height 20 cm) and fed at a flow velocity of 34 cm/h with an exosome feed which had first been diafiltered and concentrated (including DNase treatment) with TFF (cut-off 750 kDa). (Bed volume was 106 ml). FIG. 14b shows the total particle content (line) as measured with the BCA assay in different eluate fractions, and the particle concentration (bars) in different eluate fractions measured using nanoparticle tracking analysis (NTA) (ZETAVIEW® from Particle Metrix).

### DETAILED DESCRIPTION

Below is described in more detail a size exclusion chromatography method that can be used for purifying enveloped viruses and extracellular vesicles, EVs, such as exosomes. The SEC test columns used comprised a stationary phase comprising a packed bed of cross-linked agarose beads, herein called a highly cross-linked agarose packed bed. The method may be used in large-scale purification of enveloped viruses/EVs with a low level of contaminants and which does not harm the enveloped viruses/EVs.

### EXPERIMENTAL

Columns with Highly Cross-Linked Agarose Backed Bed

Agarose beads having an agarose content of 0.5-20 wt. % were used as the material of the packed bed of the chromatography column (80-95.5 wt. % of the beads being water and ethanol). The agarose bead size D50v was 75 μm and the exclusion limit at least 30 nm. The agarose beads used were cross-linked as described elsewhere herein for improved rigidity to allow for higher flows through the column. HiScreen™ columns (from Cytiva®) were used. The columns having a bed dimension of 7.7 mm×100 mm (bed volume 4.7 ml). The HiScreen™ columns were packed with agarose beads in a HiScreen™ packing machine according packing instructions for HiScreen™. HiScale™ columns (from Cytiva®), bed dimensions 26 mm×200 mm (bed volume 106 ml), and Tricorn™ columns (from Cytiva®) bed dimensions 10 mm×200 mm (bed volume 16 ml) were also used. The HiScale and Tricorn columns were packed according to manufacture instruction. The packed columns had a critical pressure of 3 bar, a max packing pressure of 2 bar, and a max operating pressure of 1.5 bar.

Reference Columns with Sepharose® CL-2B Packed Bed

Columns packed with Sepharose® CL-2B resin (Cytiva®), a bead-formed cross-linked agarose-based gel matrix (2% agarose), bead size D50v of 90 μm, were used as reference. A HiScreen™ column (from Cytiva®) was used. The column having a bed dimension of 7.7 mm×100 mm (bed volume 4.7 ml). The HiScreen™ columns were packed with Sepharose® CL-2B agarose beads in a HiScreen™ packing machine according packing instructions for HiScreen™. The packed columns had a critical pressure of 0.3 bar, a max packing pressure of 0.2 bar, and a max operating pressure of 0.15 bar.

### Size Exclusion Chromatography

The chromatography system used was an ÄKTA™ pure 25 (from Cytiva®) with a flow rate up to 25 mL/min.

### HiScreen™ Column (Bed Volume 4.7 ml)

One or two columns in series were used in the size exclusion chromatography tests. The flow rate used was 129 cm/h (1 ml/min). Sample injection volume was 500 μl. The running buffer was 10 mM PBS, pH 7.4. UV detection at 490, 280 and 214 nm.

### HiScale™ Column (Bed Volume 106 ml)

Flow velocity used was 34 cm/h. Sample injection volume was 12 ml. The running buffer was 10 mM PBS, pH 7.4. UV detection at 280 nm.

### Tricorn™ (Bed Volume 17 ml)

Flow velocity used was 38 cm/h (0.5 ml/min). Sample injection volume was 2 ml. The running buffer was 10 mM PBS, pH 7.4. UV detection at 280 nm.

### Test Solutions

### Latex Particles

Latex particles having a diameter of 40 and 60 nm were used (0.1% in 10 mM PBS, pH7.4).

### Thyroglobulin

Thyroglobulin (Sigma®), 1 mg/ml in 10 mM PBS, pH7.4.

### Albumin

Albumin (Sigma®), 1 mg/ml in 10 mM PBS, pH7.4.

### GFP-His

Green fluorescent protein with his tag (GFP-His) expressed in E. coli was purified in house. (concentration 1 mg/ml).

### Exosomes

Solutions containing exosomes from three different sources were used: 1) exosomes from the cell line HEK 293 (human embryonic kidney), 2) GFP (green fluorescent protein) immobilized exosomes (from Evox Therapeutics, eGFP harvest material), and 3) dental pulp stem cells containing exosomes (VivaZome™ Therapeutics).

### Tangential Flow Filtration (TFF)

An ÄKTA Flux S TFF system (Cytiva®) was used at a flow of 36 rpm and a transmembrane pressure (TMP) of 0.4 bar. The mixer speed was set at 50 rpm. A water flux test was performed before and after each TFF run. The TFF membrane used for HiScreen applications was: Hollow fiber Cartridge Model: UFP-750-C-2U (Cytiva®). Cutoff: 750 kDa. Area: 50 cm². The TFF membrane used for Tricorn and HiScale applications was: UFP-750-E-H42LA (73 cm2 membrane area; 1 mm id lumen)

The following TFF protocol was used:
1. Flux test
2. Concentration 10×
3. Wash with buffer 5×/diafiltration
4. Optional—Add DNA nuclease, e.g. benzonase, and recirculate for 2 h
5. Optional—Wash with buffer 5×/diafiltration
6. Optional—Concentration 10×
7. Collect retentate/sample
8. Cleaning filter
9. Flux test
10. Storage buffer

In step 4: Add 2 kU DNA nuclease, e.g. benzonase, and 1 mM MgCl₂ into the tank and note the time. (2 kU/23 ml=86.96 U/ml was added, Add 0.25 mL MgCl₂).

### ELISA Analysis

Enzyme-linked immunosorbent assay (ELISA) was used to detect membrane proteins on the surface of the exosomes. Two different ELISA kits were used: CD63: Human CD63 ELISA kit CD63 (#EH95RB, Invitrogen), and CD81: ExoELISA-ULTRA Complete Kit (CD81 detection) #EXEL-ULTRA-CD81-1, System Biosciences). The CD63 and CD81 ELISAs were performed according to manufacture protocols.

### Total Protein

BCA (bicinchoninic acid) assay (Pierce™ BCA Protein Assay Kit (#23225 Thermo Scientific)) was used. The BCA assay, measurement of total protein concentration in sample, were performed according to manufacture protocol.

### Nanoparticle Tracking Analysis (NTA)

NTA (ZETAVIEW® from Particle Metrix) was used to measure particle size and concentration according to manufacture protocol for particles in sizes between 50 and 200 nm.

### Absorbance

UV280, 490 and 214 nm was analyzed online with ÄKTA pure 25 UV detector U9-M module using Unicorn 7.7 for analysis of data (Cytiva®).

### Results

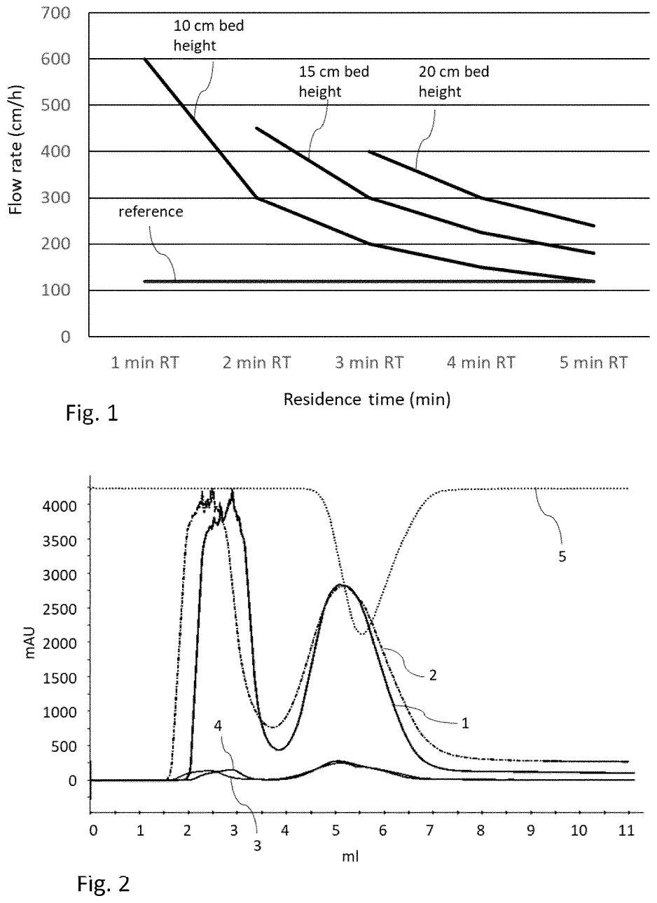

Residence times obtained for size exclusion chromatography (SEC) columns of different bed-heights run at different flow velocities (100-700 cm/h) were tested. Three different bed heights (10 cm, 15 cm and 20 cm) of columns having a stationary phase comprising a packed bed of highly cross-linked agarose beads were compared. As a reference, a column comprising a packed bed of Sepharose® CL-2B with a bed-height of 10 cm was used. The reference column having a flow limit of 120 cm/h. In FIG. 1 is shown the flow velocity vs residence times for the three test columns and the reference column. The solvent run on the columns was water.

The new highly cross-linked agarose SEC-resin allows for running of columns with a bed-height of up to 20 cm with high flow (>120 cm/h) without exceeding 3 bar (std limit to run in production environment). Short residence time is important for quick processing and opportunity to divide the sample and run several chromatography cycles and thereby reducing the column volume needed. From these results, it can be concluded that the flow and pressure properties of the highly cross-linked agarose SEC-resin, as compared to Sepharose CL-2B resins, are advantageous for efficient purification of EVs (for up-scaling and high flow rates.)

Next, the eluate from a SEC column (10 cm bed-height) comprising the highly cross-linked agarose stationary phase was compared with the eluate from a SEC column comprising a Sepharose® CL-2B stationary phase (10 cm bed-height), see FIG. 2. The columns were loaded with 1 ml solution volume (22% of bed volume) comprising a mixture of latex particles (60 nm) and proteins (GFP-His, 28 kD) at a flow velocity of 129 cm/h. The eluates from the highly cross-linked agarose packed bed and the Sepharose® CL-2B packed bed were evaluated with absorbance spectroscopy A218 nm, curve 1 and curve 2, respectively (latex particles constituting the first peak at 2-4 ml and GFP-His, the second peak at 4-7 ml). The eluate was also evaluated with absorbance spectroscopy A490 nm, curve 3 and curve 4, respectively (latex particles constituting the first peak at 2-4 ml and GFP-His constituting the second peak at 4-7 ml). (Curve 5 being the conductivity.) As can be seen, there was a better separation of the two peaks with latex particles and GFP-His, using the highly cross-linked agarose bead-containing SEC column as compared to the column with Sepharose® CL-2B.

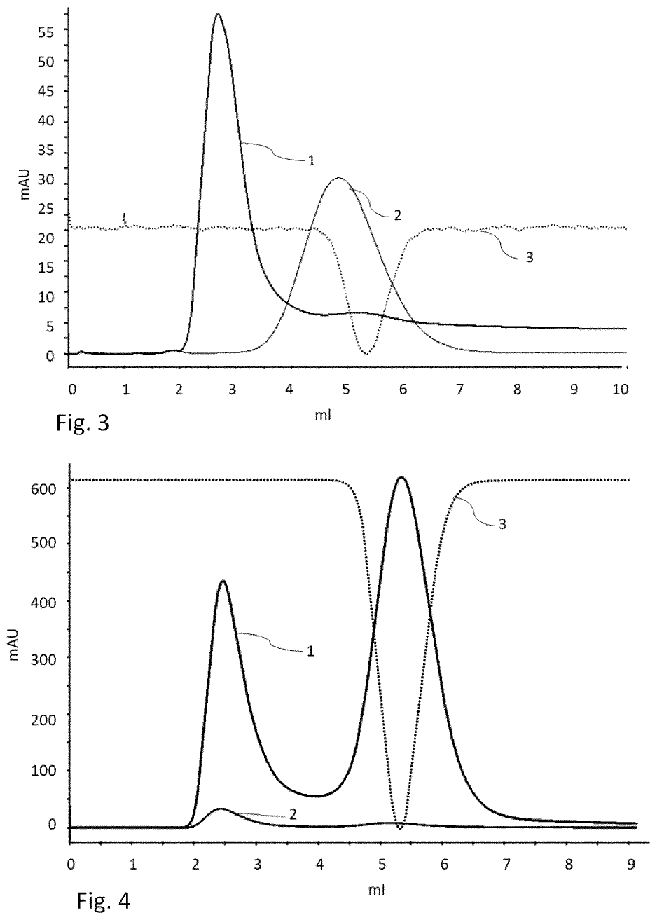

FIG. 3 shows an overlay of two chromatograms obtained from a SEC column comprising a highly cross-linked agarose stationary phase (bed height 10 cm) being loaded with solution volumes comprising latex particles (60 nm) and albumin (69 kD, 3.8 nm), respectively, at a flow velocity of 129 cm/h. The eluate was evaluated with absorbance spectroscopy A280 nm (curve 1) and A490 (curve 2) (curve 3 being the conductivity). Curve 1 shows latex particles (at 2-4 ml) and curve 2 shows albumin (at 4-7 ml). As can be seen, there is a separation between latex particles and albumin with a slight overlap.

The same SEC column as was used above was loaded with 0.5 ml of a solution volume (11% of bed volume) comprising a mixture of GFP-exosomes spiked with thyroglobulin (669 kDa) at a flow velocity of 129 cm/h. The eluate from the highly cross-linked agarose packed bed was evaluated with absorbance spectroscopy A280 nm (curve 1) and A490 nm (curve 2), see the chromatogram in FIG. 4. Curve 1 shows GFP-exosomes in the first peak (at 2-4 ml) and thyroglobulin in the second peak (at 3.5-7 ml). As can be seen, there is a separation between GFP-exosomes and thyroglobulin. Curve 2 shows that GFP-exosomes were eluted in the first peak (2.5 ml). (Curve 3 being the conductivity.)

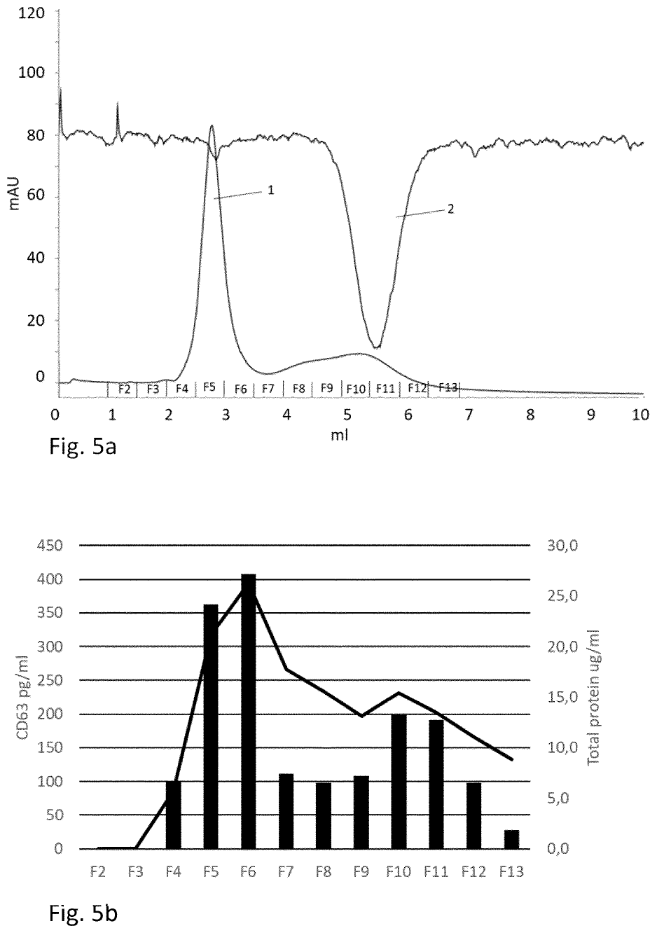

Thereafter it was tested how diafiltering and concentration of the exosome feed would influence the separation result. The feed was first diafiltered and concentrated 10-fold with tangential flow filtration (TFF) with a cut-off of 750 KDa before running the solution, sample load 0.5 ml, on the highly cross-linked agarose-bead SEC column (1× HiScreen (4.7 ml, 10 cm bed height)) at a flow velocity of 129 cm/h. FIG. 5a shows a chromatogram obtained using absorbance spectroscopy A280 nm (curve 1). (Curve 2 being the conductivity.) In FIG. 5b is shown the detection of exosomes in different eluate fractions from such a run using ELISA CD63 (marker for exosomes) detection (line) and using bicinchoninic acid assay (BCA assay) (bars).

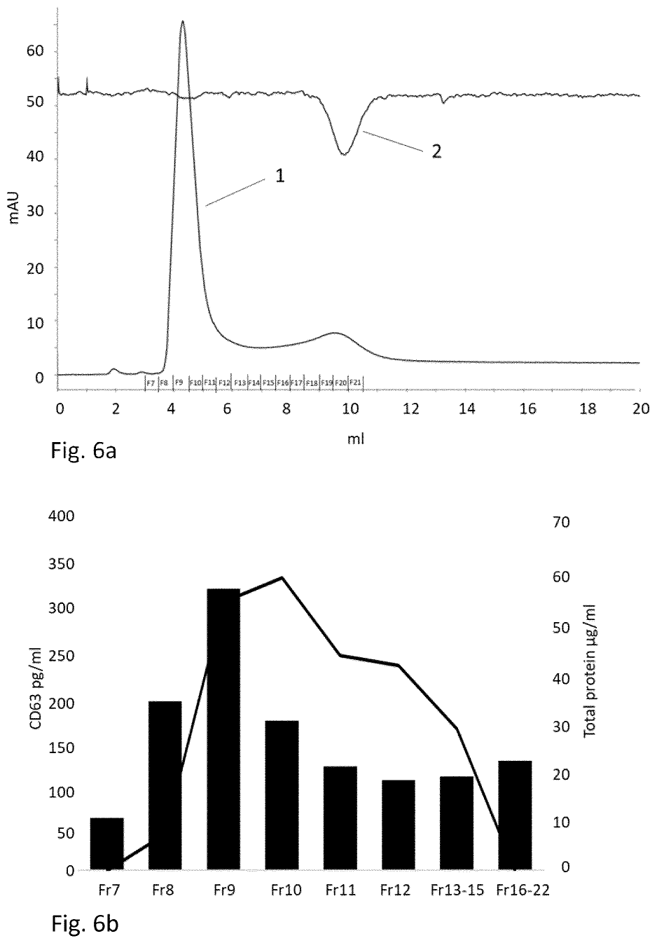

FIG. 6a shows a chromatogram with the absorbance spectroscopy result at A280 nm of an exosome feed first diafiltered and concentrated 10-fold with tangential flow filtration (TFF) with a cut-off of 750 KDa and thereafter run through a size exclusion column comprising an agarose stationary phase with a bed-height of 20 cm (2× HiScreen (9.4 ml, 20 cm bed height)) and at a flow velocity of 129 cm/h, curve 1. (Curve 2 being the conductivity.) FIG. 6b shows detection of exosomes in different eluate fractions using ELISA CD63 detection (line) and BCA assay (bars). Comparing the results shown in FIGS. 5a and 5b with those in FIGS. 6a and 6b, it was shown that the separation was improved with a bed height of 20 cm as compared to a bed-height of 10 cm.

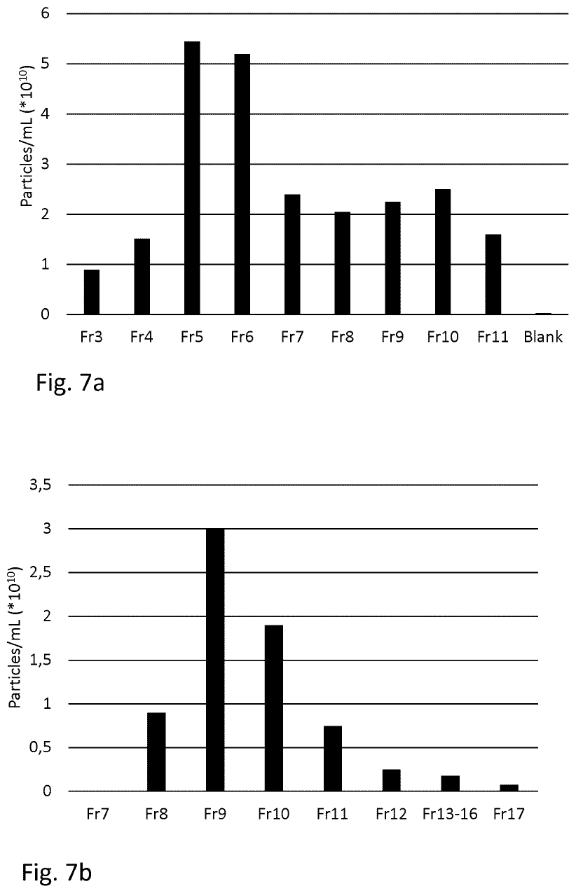

FIGS. 7a and 7b show the particle concentration in different fractions eluted from the columns discussed for FIGS. 5a-5b and FIGS. 6a-6b, respectively. The concentration was measured using nanoparticle tracking analysis (NTA) (ZETAVIEW® from Particle Metrix). Particle concentrations confirm the CD63 data in FIGS. 5b and 6b.

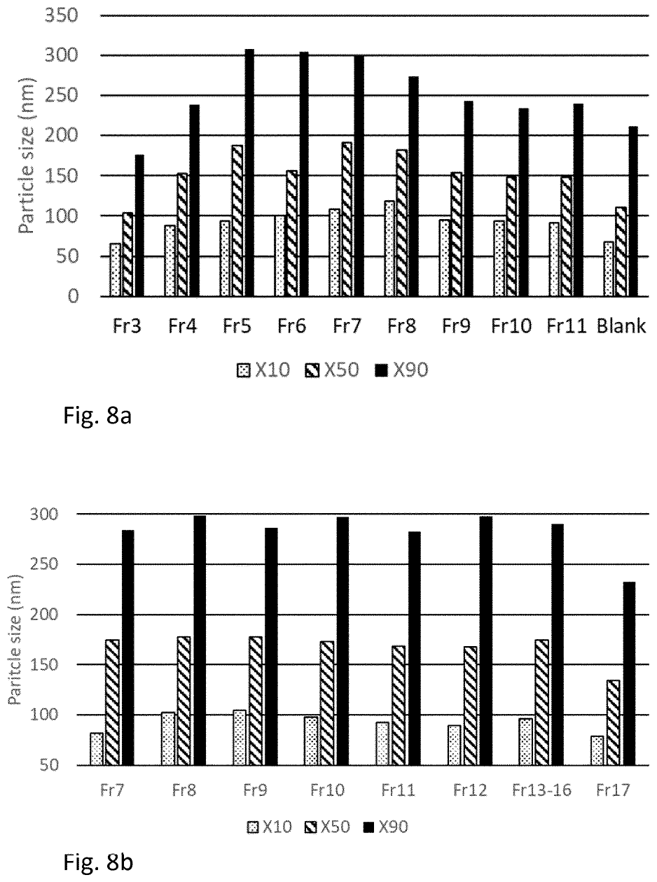

FIGS. 8a and 8b show the particle size in different fractions eluted from the columns discussed for FIGS. 5a-5b and FIGS. 6a-6b, respectively. The particle size was measured using nanoparticle tracking analysis (NTA) (ZETAVIEW® from Particle Metrix). X10=particle size where 10% of the particles are smaller than that particle size (nm), X50=particle size where 50% of the particles are smaller than that particle size (nm) and X90=particle size where 90% of the particles are smaller than that particle size (nm). The size distribution measured by NTA did not differ between the fractions from a column with a 20 cm column bed height, whereas for a column with a 10 cm bed height, the size distribution varied slightly.

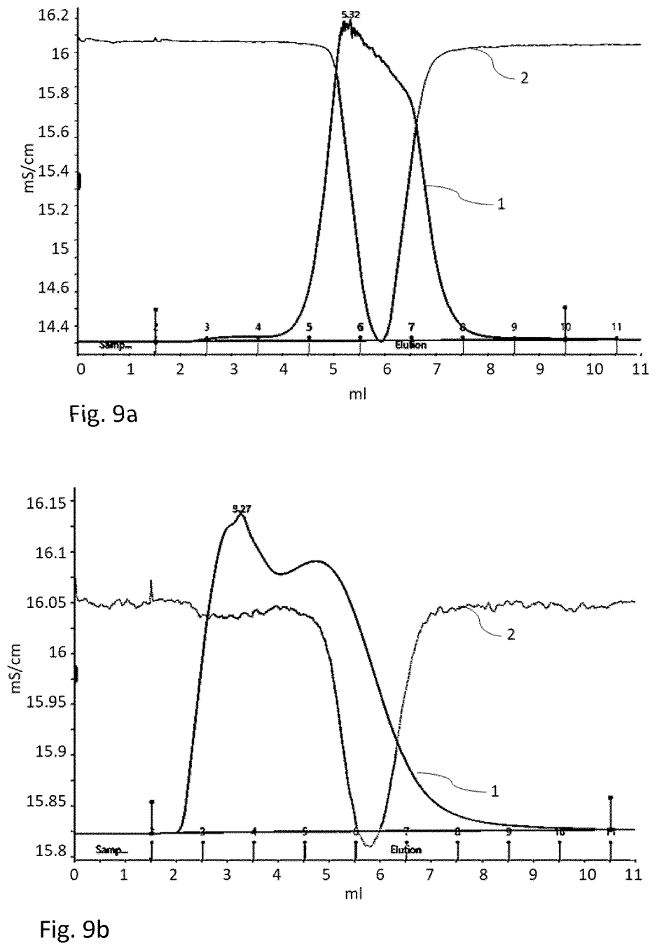

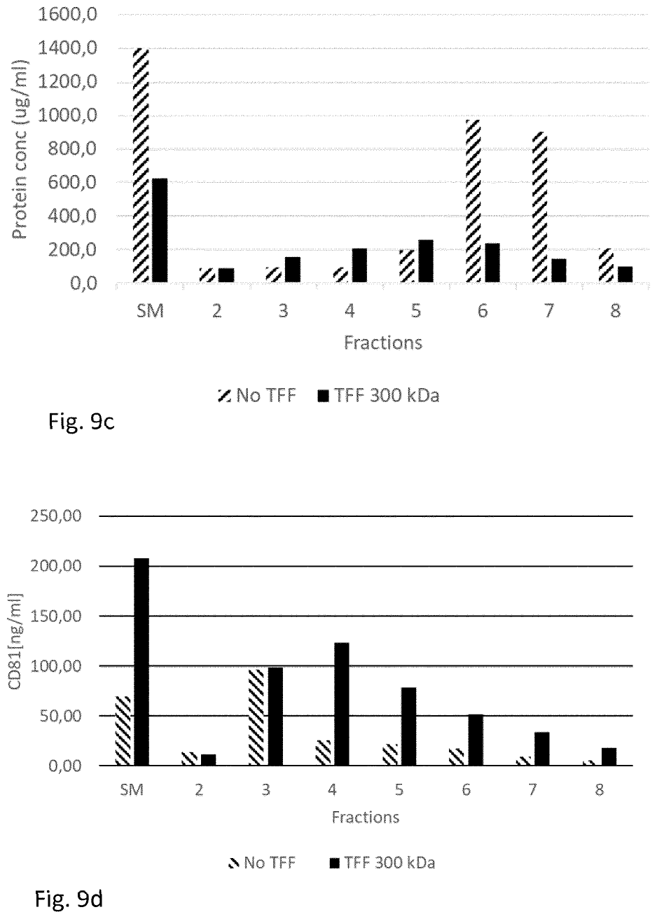

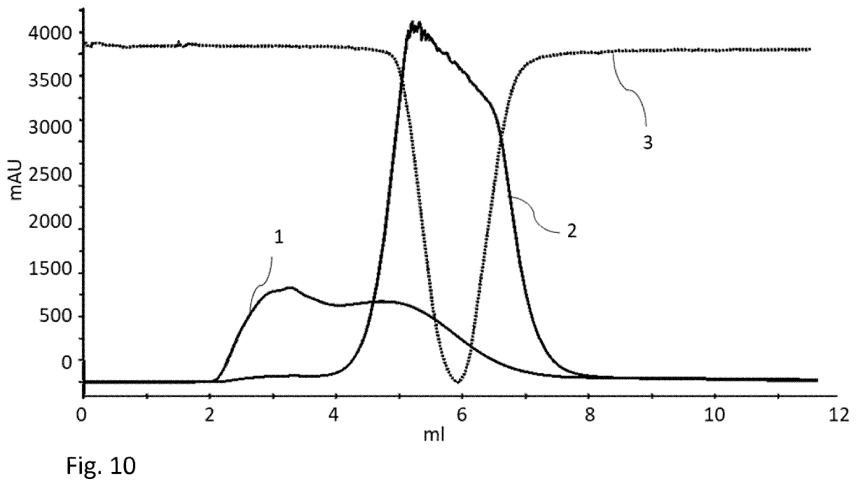

Next, a harvest comprising exosomes produced by a HEK (Human Embryonic Kidney) 293 cell line was tested. FIG. 9a shows a chromatogram obtained from a SEC column with a packed bed of highly cross-linked agarose beads (bed height 10 cm) and fed at a flow velocity of 126 cm/h with such a harvest, as measured with A280 nm, curve 1. (Curve 2 being the conductivity.). FIG. 9b is a chromatogram obtained from a SEC column with a packed bed of highly cross-linked agarose beads (bed height 10 cm) and fed at a flow velocity of 129 cm/h with a harvest comprising exosomes produced by a HEK (Human Embryonic Kidney) 293 cell line, which harvest before being applied to the SEC column had been diafiltered and concentrated 10-fold with a TFF with a cut-off of 750 KDa. In FIG. 9c is shown the total protein concentration in different fractions eluted from the columns discussed for FIGS. 9a (no TFF) and 9b (with TFF) using the BCA assay. In FIG. 9d is shown the exosome content in different fractions eluted from the columns discussed for FIGS. 9a (no TFF) and 9b (with TFF) using the ELISA CD81 assay. From these experiments it can be concluded that a TFF step, for protein removal and exosome concentration, prior to running the exosome harvest on the highly cross-linked agarose packed-bed SEC gives the possibility to load less volume on the SEC column. Contaminants remaining after the TFF step, such as proteins, can be removed by the SEC. From FIGS. 9a-9d it is seen that the SEC agarose column with highly cross-linked agarose beads can separate large entities from proteins, and that TFF improves the separation. This is further illustrated in FIG. 10, which shows an overlay of the graphs shown in FIG. 9a and FIG. 9b. Curve 1 is the graph from FIG. 9b and curve 2 is the graph from FIG. 9a. (Curve 3 being the conductivity.)

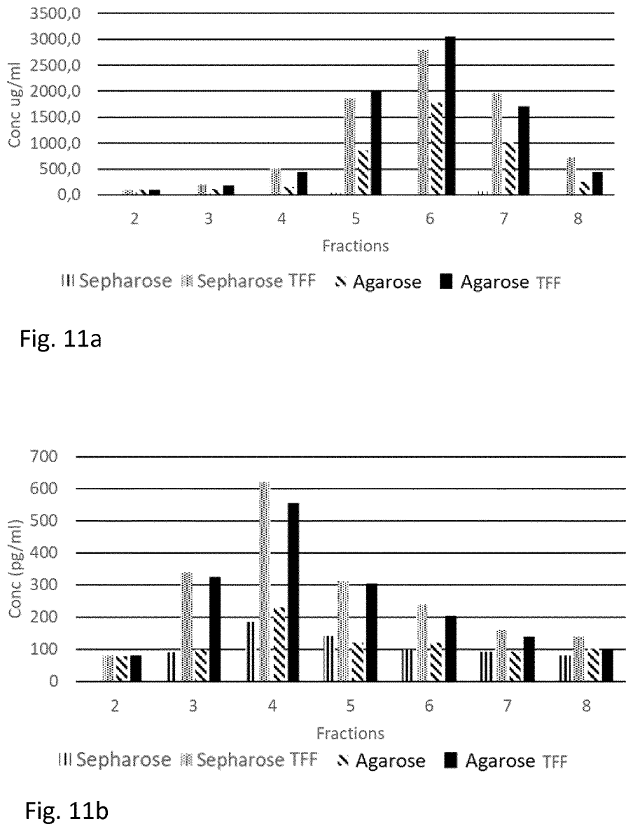

FIG. 11a shows the total protein content as measured with the BCA assay and in FIG. 11b is shown the exosome content as measured with ELISA CD63 in different eluate fractions from highly cross-linked agarose SEC columns and columns with Sepharose® CL-2B, respectively (bed height 10 cm). The columns being fed at a flow velocity of 129 cm/h with a harvest comprising exosomes produced by a dental pulp cell line. The graph shows the result with and without a preceding diafiltering step and 10× concentration with TFF with a cut-off of 750 kDa. The sample (harvest) was treated with DNase (benzonase) before being filtered and concentrated in the TFF step. Using TFF in combination with highly cross-linked agarose SEC gives improved enrichment of exosome fractions and removes most of host proteins.

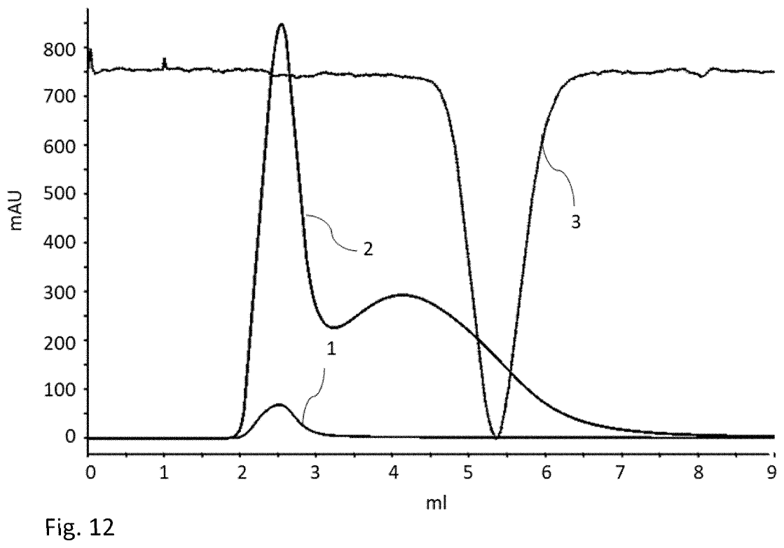

FIG. 12 shows a chromatogram obtained from a highly cross-linked agarose SEC column (10 cm bed height) loaded at a flow velocity of 129 cm/h with a sample containing GFP-exosomes spiked with thyroglobulin (680 kDa) as measured with A490 nm and A280 nm. Exosomes and thyroglobulin were separated. Curve 1 was measured with A490 nm, which detects GFP in the exosomes. Curve 2 was measured with A280 nm and detects both GFP exosomes and thyroglobulin. (Curve 3 being the conductivity.) The chromatogram shows that larger proteins such as Thyroglobulin (700 kDa (^{~}15 nm)) can be removed from exosomes.

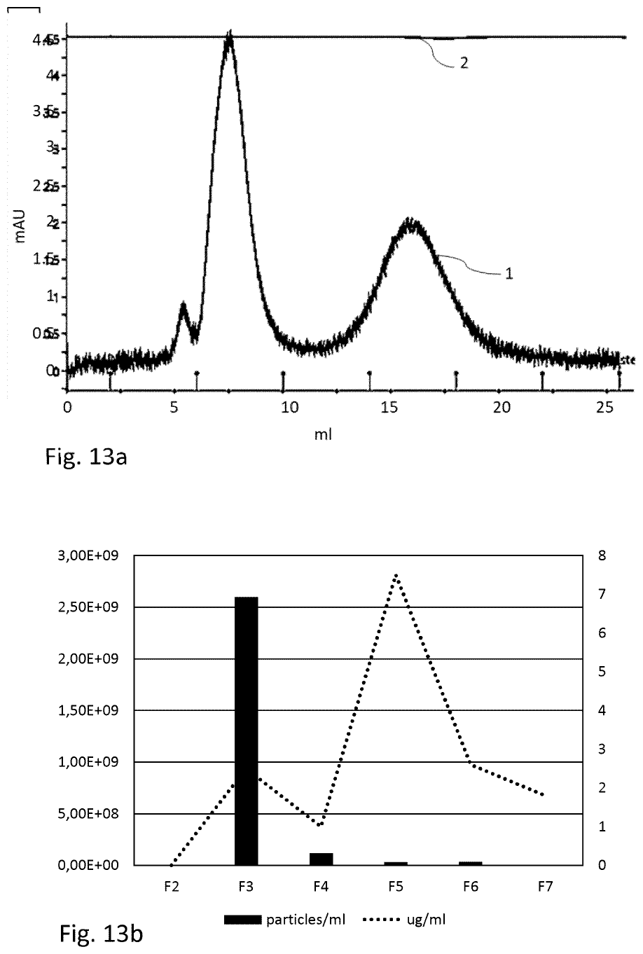

Next, up-scaling of the SEC process was evaluated with a highly cross-linked agarose bead column having a bed volume of 17 ml (Tricorn) and a bed height of 20 cm. FIG. 13a shows the absorbance spectroscopy result at A280 nm of an exosome feed (Dental pulp stem cells (Vivazome) first diafiltered and concentrated (treated with DNase) 10-fold with tangential flow filtration (TFF) with a cut-off of 750 KDa and thereafter run through the SEC column at a flow velocity of 38 cm/h, curve 1. (Curve 2 being the conductivity.) FIG. 13b shows the total particle content (line) in different eluate fractions as measured with the BCA assay, and the particle concentration (bars) in different eluate fractions, as measured using nanoparticle tracking analysis (NTA) (ZETAVIEW® from Particle Metrix). The results show that proteins that were not removed by TFF could be separated by SEC.

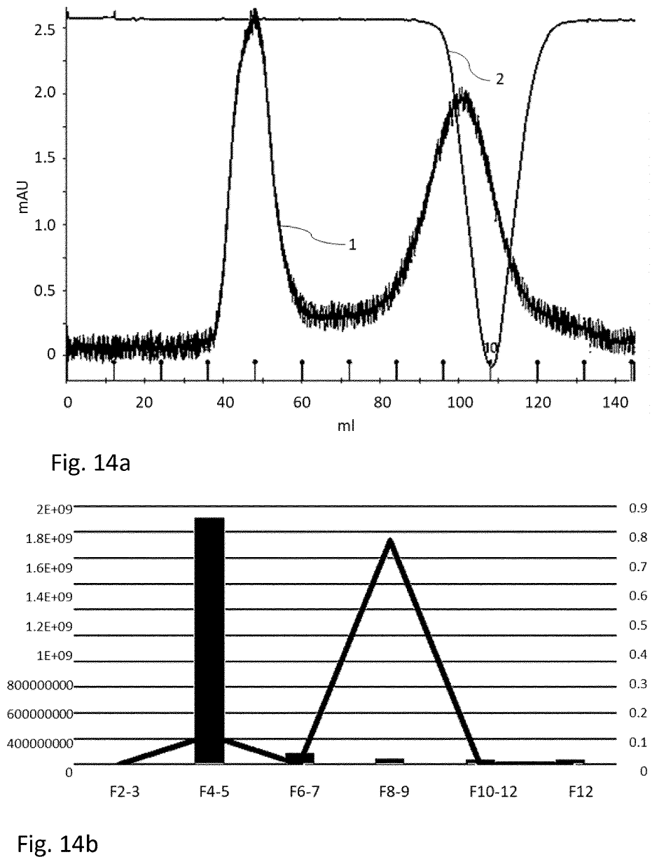

In yet an up-scaling test, FIG. 14a shows a chromatogram obtained from a highly cross-linked agarose SEC column (bed-height 20 cm, HiScale™ column (from Cytiva®), bed dimensions 26 mm×200 mm, bed volume 106 ml) and fed at a flow velocity of 34 cm/h with an exosome feed that had first been diafiltered and concentrated 10-fold with TFF (cut-off 750 kDa) as measured with A280 nm, curve 1. (Curve 2 being the conductivity.) FIG. 14b shows the total particle content (line) as measured with the BCA assay in different eluate fractions, and the particle concentration (bars) in different eluate fractions measured using nanoparticle tracking analysis (NTA) (ZETAVIEW® from Particle Metrix). FIGS. 13a-14b show that it is possible to scale up the highly cross-linked agarose SEC column while maintaining the good separation between exosomes and proteins. The lower flow velocity compared to that used for FIGS. 2-12 improved the separation as well. Exosome particles were eluted in mostly two fractions and proteins elute after the exosomes. In Table 1 below is summarized the results and recovery data from the experiments discussed and shown in FIGS. 13a, 13b, 14a and 14b.

TABLE 1

| | Sample | | | Total | Particle | |
|---|---|---|---|---|---|---|
| | load | EV | Fraction | protein | conc | Recovery |
| Column | (ml) | Fractions | volume (ml) | (ug/ml) | (part/ml) | (%) |
| Tricorn | 2 | F3 | 4 | 2.5 | 2.60E+09 | 49 |
| 10/200 | | | | | | |
| HiScale | 12 | F4-F5 | 24 | 0.12 | 1.90E+09 | 69 |
| 26/200 | | | | | | |

Enveloped viruses with sizes above 30 nm would follow the same separation pattern as exosomes and latex particles, as shown above, since they have similar hydrodynamic radius as exosomes and are more or less spherical.

A size-exclusion chromatography column having a packed bed comprising the highly cross-linked agarose beads and having a height of 20 cm and a diameter of 60 cm was also prepared. In Table 2 below is shown pressure drop (bar) and flow velocities (cm/h) for such a packed bed.

TABLE 2

| Flow velocity (cm/h) | Pressure (bar) |
|---|---|
| 30 | 0.1 |
| 170 | 0.3 |
| 200 | 0.4 |
| 260 | 0.5 |
| 310 | 0.6 |

As can be seen from the table, the pressure drop is below 1 bar for a 60 cm diameter column packed to 20 cm bed height with the highly cross-linked agarose beads at flow rates up to 300 cm/h. Hence, it is seen that the highly cross-linked beads are compatible with packing in large-scale columns and suitable for chromatography at flow-rates of several hundred cm per hour.

1. Method of purifying extracellular vesicles or enveloped viruses, comprising:
obtaining a solution volume comprising extracellular vesicles or enveloped viruses and contaminants,
adding the solution volume to a size exclusion chromatography column comprising a stationary phase comprising a packed bed of cross-linked polysaccharide beads,
collecting an eluate volume comprising the extracellular vesicles or enveloped viruses exiting the column.

2. The method of claim 1, further comprising, before adding the solution volume comprising extracellular vesicles or enveloped viruses to the size exclusion chromatography column, a step of filtering the solution volume comprising the extracellular vesicles or enveloped viruses with a filter or membrane to remove contaminants being of a size smaller than the extracellular vesicles or enveloped viruses from the sample volume.

3. The method of claim 2, further comprising concentrating the filtered solution volume.

4. The method of claim 2, further comprising, before or after filtering the solution volume, adding deoxyribonuclease to the solution volume.

5. The method of claim 2, wherein filtering is performed with a filter or membrane with a cut-off of 750 kDa.

6. The method of claim 1, wherein the solution volume is added to the size exclusion chromatography column at a flow velocity of at least 30 cm/hour.

7. The method of claim 1, wherein the solution volume is added to the size exclusion chromatography column at a pressure of at most 3 bar.

8. The method of claim 7, wherein the solution volume comprising extracellular vesicles or enveloped viruses is added to the column in a ratio of solution volume to column/packed bed volume of up to 1:3.

9. The method of claim 1, wherein the packed bed of polysaccharide beads has a height of 2.5 to 60 cm, such as 10 to 60 cm.

10. The method of claim 1, wherein the chromatography column has a width of 0.5 cm to 70 cm in diameter.

11. The method of claim 1, wherein the extracellular vesicles or enveloped viruses are exosomes.

12. The method of claim 1, wherein the cross-linked polysaccharide beads are agarose beads.

13. The method of claim 12, wherein the agarose beads have an agarose content of 0.5-20 wt. %.

14. The method of claim 13, the agarose beads have an agarose content of >2-20 wt. %, such as 2.5-20 wt. %.

15. The method of claim 1, wherein the cross-linked polysaccharide beads have an exclusion limit of at least 30 nm.
